(19) United States
(12) Patent Application Publication
(10) Pub. No.: US 2006/0024249 A1
(43) Pub. Date: Feb. 2, 2006

(54) Methods and compositions for bioengineering a tooth

(76) Inventors: Pamela C. Yelick, Concord, MA (US); Joseph P. Vacanti, Winchester, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/183,451

(22) Filed: Jul. 18, 2005

Related U.S. Application Data
(60) Provisional application No. 60/588,662, filed on Jul. 16, 2004.

(51) Int. Cl. C12N 5/08; A61K 8/96

(52) U.S. Cl. 424/50; 435/366

(57) ABSTRACT

This invention relates generally to methods and compositions for bioengineering tooth tissue, as well as methods of producing new tooth tissue in a subject.

### RELATED APPLICATIONS

This application claims the benefit of U.S. Provisional Application No. 60/588,662, filed Jul. 16, 2004, the entire contents of which is hereby incorporated by reference.

### FIELD OF THE INVENTION

This invention relates generally to methods and compositions for bioengineering tooth tissue, as well as methods of producing new tooth tissue in a subject.

### BACKGROUND OF THE INVENTION

The incidence of children born with missing primary and/or adult teeth is significant, and tooth loss in aged populations is also a prevalent health problem. In addition, there are genetically inherited tooth defects, such as enamel defects (e.g., amelogenesis imperfecta) and/or dentin defects (e.g., dentinogenesis imperfecta).

Current replacement tooth methods to treat these and other tooth defects use synthetic materials that do not possess the characteristics of natural teeth, such as the ability to respond to the environment by migrating to maintain a proper bite. In addition, these synthetic materials can elicit an immune response in a host subject.

Thus, a significant need exists for replacement teeth. The practice of dentistry would be revolutionized by providing a means to replace a defective or diseased tooth with a healthy and permanent bioengineer tooth.

### SUMMARY OF THE INVENTION

The present invention provides methods and compositions for bioengineering tooth tissue, as well as methods of producing new tooth tissue in a subject.

The compositions include, for example, an isolated dental epithelial precursor cell, an isolated dental mesenchymal precursor cell or a mixture of both cell types. The dental epithelial precursor cells and dental mesenchymal precursor cells are STRO-1⁺. In a Hoechst 33324 dye profile, the dental epithelial precursor cells and dental mesenchymal precursor cells exhibit a low or negative level of Hoechst 33324 dye. The dental epithelial precursor cell differentiates into an enamel-producing cell, and the dental mesenchymal precursor cell differentiates into a dentin-producing cell.

In a composition that contains an isolated dental epithelial precursor cell, the cell is obtained from a post-natal tooth bud. In a composition that contains an isolated dental mesenchymal epithelial precursor cell, the cell is obtained from a tooth pulp tissue or a post-natal tooth bud. In a composition that includes both isolated dental epithelial precursor cells and isolated dental mesenchymal epithelial precursor cells, the cells are obtained from a tooth pulp tissue or a post-natal tooth bud.

Biocompatible implants include, for example, a biocompatible scaffold and an isolated dental epithelial precursor cell, an isolated dental mesenchymal precursor cell or a mixture of both cell types.

In a biocompatible implant that contains an acellular scaffold, a first population of isolated dental mesenchymal precursor cells, a second population of isolated dental epithelial precursor cells, the first population and the second population form an interface, but these cell populations are spatially segregated on the acellular scaffold. The interface between the first and second cell populations is, for example, a contact. Enamel is present at the interface between the first population and the second population.

A biocompatible implant includes, for example, a first biocompatible scaffold seeded with a population of isolated dental epithelial precursor cells, and a second biocompatible scaffold seed with a population of isolated dental mesenchymal precursor cells. The first and second biocompatible scaffolds are oriented in relation to each other, such that a portion or all of the first scaffold is in contact with a portion or all of the second scaffold, thereby forming an interface between the scaffolds. For example, the first biocompatible scaffold is located within the biocompatible implant at a position that is inferior to the location of the second biocompatible scaffold, such that at least a portion of said interface between the scaffolds produces a dentin/enamel junction.

Methods for generating tooth tissue in a subject involve implanting a biocompatible that includes a biocompatible scaffold and a population of isolated dental epithelial precursor cells, a population of isolated dental mesenchymal precursor cells or a mixture of both cell types at a vascularized site in a subject. Vascularized sites of implantation include, for example, highly vascularized sites such as the omentum, the eye and the renal capsule of a subject. Suitable subjects include mammals.

### BRIEF DESCRIPTION OF THE FIGURES

FIGS. 1A-1D are a series of photographs depicting STRO-1 positive (STRO-1⁺) cultured dental cells. Panel A depicts the expression of STRO-1 in cultured epithelial and mesenchymal pig tooth bud cells (as indicated by the arrows), and Panel C depicts the expression of STRO-1 in cultured epithelial and mesenchymal rat tooth bud cells (as indicated by the arrows). Panels B and D depict isotype matched negative controls for Panels A and C, respectively.

FIG. 2 is a plot depicting Hoechst dye profiling of primary rat tooth bud cell cultures in which the cells were sorted based on their forward and side light scatter characteristics. The side population (SP) cells are shown in the boxed region in FIG. 2.

FIGS. 3A and 3B are photographs depicting cloned dental epithelial and mesenchymal side population cells after two weeks of culture.

FIGS. 4A-4C are a series of photographs depicting one embodiment of a biocompatible implant according to the invention. Panel A depicts the presence of 5 mm diameter tooth tissue in the cross-section of a paraffin-embedded implant. Panel B depicts a high magnification image of the boxed region in Panel A, which is bioengineered Hertwig's epithelial root sheath (hers), a distinct morphological feature of early tooth root formation. Panel C depicts a high magnification image of the presence of odontoblasts (od) and bioengineered tubular dentin (d) in the tooth tissue.

FIGS. 5A and 5B are a series of photographs depicting a bioengineered dentin/enamel junction (DEJ) in one embodiment of a biocompatible implant that includes two distinct biocompatible scaffolds, where one scaffold is seed with pulp organ cells and the second is seed with enamel organ cells. Pane A shows a distinct DEJ formed at the interface of the scaffolds (see red dotted line) seeded with either pulp organ cells or enamel organ cells. Panel B is a high magnification image of the DEJ, where “d” represents the presence of distinct dentin and “e” represents the presence of demineralized enamel at the DEJ.

### DETAILED DESCRIPTION OF THE INVENTION

This invention provides methods and compositions for producing a biological tooth replacement using tissue-engineering methodology based on seeding isolated cell populations onto biocompatible scaffolds, and allowing the cell/scaffold constructs, also referred to herein as “biocompatible implants” to develop into tooth tissues inside of a suitable host. Developing tooth tissues derived from such biocompatible implants are surgically implanted into the gum of an edentulous recipient where the construct is exposed to a blood supply and develops to maturity, providing the recipient with a biological tooth replacement. A biological tooth substitute that is properly formed and integrated into the jaw of a human patient outlasts synthetic dental implants since a living tooth responds to its environment by migrating to maintain a proper bite, and has regenerative properties in response to injury. Synthetic implants do not have these capabilities.

Tissue Engineering

Dissociated cells from a tissue or organ are used to seed biodegradable polymer scaffolds, which are implanted within a suitable host such that a sufficient blood supply allows the cells to organize into higher ordered structures around the scaffold. The maintenance of cell structures, such as those present in organs, is not possible without a blood supply. Within a matter of weeks the scaffold dissolves and the dissociated cells will have organized into a tissue or organ that was pre-determined by the size and shape of the original scaffold.

Small pieces (organoid units) or single cell suspensions of enzymatically digested tissue are seeded onto polyglycolic acid (PGA) scaffolds or other acellular scaffolds materials and incubated in culture for various times. In vitro culture prior to implantation into a host animal is minimized.

Tooth Tissue Engineering

A developing molar tooth germ is encapsulated within the jaw from which it will eventually erupt. The tooth germ is first observed as a developing bud (bud stage), which fans out into a cap-like structure (cap stage), and finally develops into a bell-like form (bell stage). It is during the late bell stage that odontoblasts and ameloblasts differentiate and deposit the organic matrices of dentin and enamel. The development of the tooth germ depends on reciprocal interactions between the epithelial and mesenchymal tissues. Epithelial-mesenchymal cell interactions are essential for developing teeth. In the tooth, mesenchymal cells form the dentin while cells of epithelial origin form the enamel. Although each mineralized tissue is formed from its respective cells of origin, epithelial-mesenchymal interactions are required to initiate the mineralization process.

Standard tissue engineering techniques using non-dental tissues have successfully demonstrated epithelial-mesenchymal interactions, e.g., in engineered intestinal tissue, and the initiation of the mineralization process, e.g., in bone and cartilage. As shown in the Examples provided below, the methods and compositions described herein generate a tissue-engineered tooth using techniques similar to those that were used successfully to generate a bioengineered intestine and a phalanges with joints.

Biocompatible implants include at least one biocompatible acellular scaffold associated with a dental cell population. The scaffolds can have a variety of shapes, but preferably, the scaffolds are molded in the shape of human teeth. The scaffold is formed using virtually any material or delivery vehicle that is biocompatible, bioimplantable, easily sterilized and that has sufficient structural integrity and physical and/or mechanical properties to effectively provide for ease of handling in a laboratory and/or surgical environment and to permit it to accept and retain sutures or other fasteners without substantially tearing. Alternatively, the scaffold is in the form of an injectable gel that would set in place at the defect site. Sufficient strength and physical properties are developed in the scaffold through the selection of materials used to form the scaffold, and the manufacturing process. Preferably, the scaffold is also pliable so as to allow the scaffold to adjust to the dimensions of the target site of implantation. In some embodiments, the scaffold can be a bioresorbable or bioabsorbable material.

Scaffolds are seeded with dental cell populations. Suitable dental cell populations include, for example, a population of purified dental epithelial precursor cells and purified dental mesenchymal precursor cell. The term “purified” cell population refers to a population of cells that is substantially free of non-dental epithelial precursor cells or non-dental mesenchymal precursor cells, cellular material or other contaminating proteins from the tissue source from which the cells are derived (e.g., post-natal tooth bud). A purified cell population contains 50% or more of the desired cells in the cell population, preferably 75% or more of the cell population, more preferably 85% or 90% or more of the cell population, and most preferably 95% or more (substantially pure) of the cell population.

As used herein, the term “dental epithelial precursor cell” refers to a cell that has one or more of the following characteristics: the cell is STRO-1⁺; the cell does not include or display a Hoechst 33324 dye in a Hoechst 33324 profile; the cell differentiates into an enamel-producing cell; and the cell differentiates into a cell that exhibits an epithelial dental cell phenotype. A “dental mesenchymal precursor cell” refers to a cell that has one or more of the following characteristics: the cell is STRO-1⁺; the cell does not include or display a Hoechst 33324 dye in a Hoechst 33324 profile; the cell differentiates into a dentin-producing cell; and the cell differentiates into a cell that exhibits a mesenchymal dental cell phenotype. The phenotype exhibited by a putative dental epithelial precursor cells or a putative dental mesenchymal precursor cell is determined, for example, by immunohistochemical staining using antibodies specific for tooth epithelial markers (e.g., keratin, amelogenin) and mesenchymal markers (e.g., osteocalcin, bone sialoprotein and dentin sialophosphoprotein). Immunofluorescence using the above markers is applied to cells in culture to characterize them prior to seeding on the biocompatible scaffold(s).

Preferably, the dental cell populations are derived from a mammalian tooth bud. Thus, the dental epithelial precursor cells and dental mesenchymal precursor cells used in the methods and compositions of the present invention are derived from post-natal tissue. The dental cell populations to be used to seed a scaffold to be implanted into an individual are preferably histocompatible, e.g., autologous cells. For example, the dental cell populations are derived from a tooth bud of a subject and preserved (e.g., frozen or immortalized) during the lifetime of the subject. Thus, the preserved autologous cell populations are useful in treating a variety of tooth injuries and defects, and in addition, the preserved autologous cell populations are useful in generating new teeth in an aged or injured subject.

The dental epithelial precursor cells and dental mesenchymal precursor cells described herein are seeded onto the biocompatible scaffold(s) using any of a variety of techniques known in the art. A population of either dental epithelial precursor cells or dental mesenchymal precursor cells is seeded on scaffold. Alternatively, a mixture of dental epithelial precursor cells and dental mesenchymal precursor cells are seeded on a single scaffold. For example, the first population of cells includes isolated dental mesenchymal precursor cells and the second population of cells contains isolated dental epithelial precursor cells, and the two populations of cells are spatially segregated on the scaffold, but they are seeded on the scaffold to produce an interface between the two populations of cells. The interface is, for example, a contact between the first and second populations of cells. In yet another embodiment, one type of dental precursor cell (e.g., dental mesenchymal precursor cells) is seeded onto a first biocompatible scaffold and a second type of dental precursor cell (e.g., dental epithelial precursor cells) is seeded onto a second biocompatible scaffold.

The biocompatible implants are implanted into a highly vascularized site in a subject so that the developing tooth tissues receive an adequate blood supply. As used herein, the term subject refers to a mammalian subject, e.g., a human or veterinary subject. Highly vascularized sites in a subject include, for example, the omentum, the eye and the renal capsule. Upon implantation, the dental precursor cells proliferate and differentiate. For example, upon implantation the dental epithelial precursor cells differentiate into enamel-producing cells and exhibit an epithelial cell phenotype, while the dental mesenchymal precursor cells differentiate into dentin-producing cells and exhibit a mesenchymal cell phenotype. The phenotype expressed by the resulting tooth tissue is analyzed using histological staining methods such as Von Kossa (calcification), Goldner's (ossification), and Van Gieson's (collagen). Immunohistochemical staining is also performed using antibodies specific for tooth epithelial markers (e.g., keratin, amelogenin) and mesenchymal markers (e.g., osteocalcin, bone sialoprotein and dentin sialophosphoprotein).

The biocompatible implant contains a mixture of dental epithelial precursor cells and dental mesenchymal precursor cells are seeded on an acellular scaffold such that the two populations of cells are spatially segregated on the scaffold, but there is an interface (e.g., contact) between the two populations of cells. Post-implantation, the dental epithelial cells differentiate into enamel-producing cells and the dental mesenchymal precursor cells differentiate into dentin-producing cells. Preferably, enamel, dentin or both are produced at the interface between the two populations.

In another embodiment, dental mesenchymal precursor cells are seeded onto a first biocompatible scaffold and dental epithelial precursor cells are seeded onto a second biocompatible scaffold. Preferably, when the biocompatible implant is implanted at a highly vascularized site in a subject, at least a portion of the first scaffold is in direct contact with a portion of the second scaffold. More preferably, the first biocompatible scaffold is implanted at a site in the subject that is inferior to the site at which the second biocompatible implant is implanted. As used herein, the term “inferior” is defined for any given point in relation to a second point that is “superior”, wherein the “inferior” point is located lower than, or below, the “superior” point. Post-implantation, the portion of the first and second scaffolds in contact, also referred to herein as the interface between the two scaffolds, produce a dentin/enamel junction (DEJ) that is marked by the presence of adjacent layers of dentin and enamel.

The invention is further described in the following examples, which do not limit the scope of the invention described in the claims.

### EXAMPLES

### Example 1

### Identification and Cloning of Dental Precursor Cells

Expression of STRO-1 in Cultured Epithelial and Mesenchymal Dental Cells

Tissue samples were taken from pig and rat post-natal tooth buds. The tissue samples were minced to create tooth bud cell suspensions. Optionally, the minced tissue fragments are further subject to enzymatic dissociation, e.g., using collagenase, to produce single cell suspensions. The cell suspensions were cultured using standard cell culture techniques.

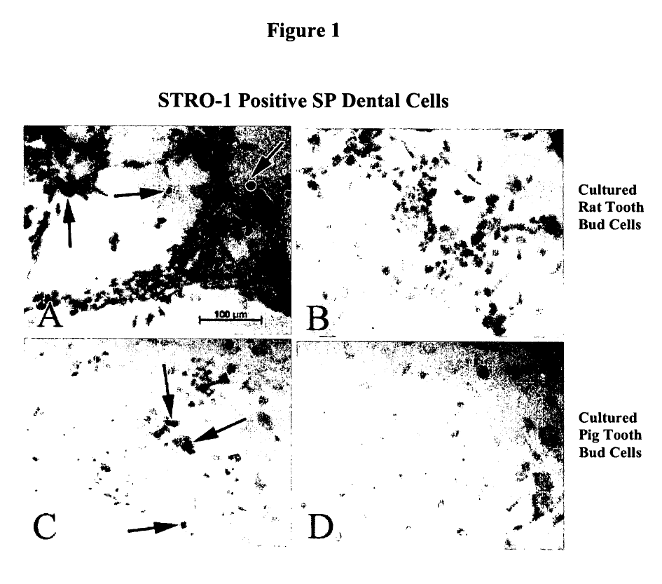

Immunohistochemical analysis of the cultured pig and rat tooth bud cells was performed using a monoclonal antibody to STRO-1 (The Developmental Studies Hybridoma Bank, Iowa City, Iowa), a surface marker for bone marrow stem cells. The presence of STRO-1 positive (STRO-1⁺) cells was used a marker for identifying dental precursor cells in the cultured tooth bud cells. The results of this immunohistochemical analysis are shown in FIGS. 1A-1D, where FIG. 1A depicts the expression of STRO-1 in cultured epithelial and mesenchymal pig cells and FIG. 1C depicts STRO-1 expression in cultured epithelial and mesenchymal rat cells (FIGS. 1B, 1D depict isotype matched negative controls). Approximately 5-10% of 14 day cultured pig tooth bud cells and 7 day cultured rat tooth bud cells were identified as being STRO-1⁺ cells, and both dental epithelial and mesenchymal STRO-1⁺ cells were identified in these rat and pig cells. These results indicate that the pig and rat dental precursor cell populations are maintained and propagated in vitro. Thus, post-natal tooth bud cells are used to bioengineer mammalian tooth tissues.

Identification of Dental Cell Side Populations

Tooth bud tissue was dissected and processed to produced a suspension of single cells. Hoechst 33342 profiling, also referred to herein as Hoechst dye profiling, was used to identify side populations (SPs), i.e., enriched stem cell populations, in a variety of tissues using known methods, e.g., Preffer et al., Stem Cells, 20(5):417-27 (2002). The target cells are identified and purified using cell sorting based on a Hoechst dye profile. A Hoechst dye profile is a method that labels a population of cells with a detectable label. As used herein, the term “detectable label” refers to a cell-permeable DNA binding dye such as Hoechst 33342. In a Hoechst 33342 dye profile, the population of cells is analyzed on a flow cytometer equipped with an ultraviolet laser, as Hoechst 33342 emits primarily in the blue range (around 450 nm) but also has a weaker red emission component. When these two emission wavelengths are detected for a population of cells and plotted against each other, a “side population” of cells, which do not include or display the detectable label, is distinguished from the remaining, labeled cells in the population. This side population of cells has been shown to be highly enriched for stem cells in a variety of cells, such as, neural cells and hematopoietic cells.

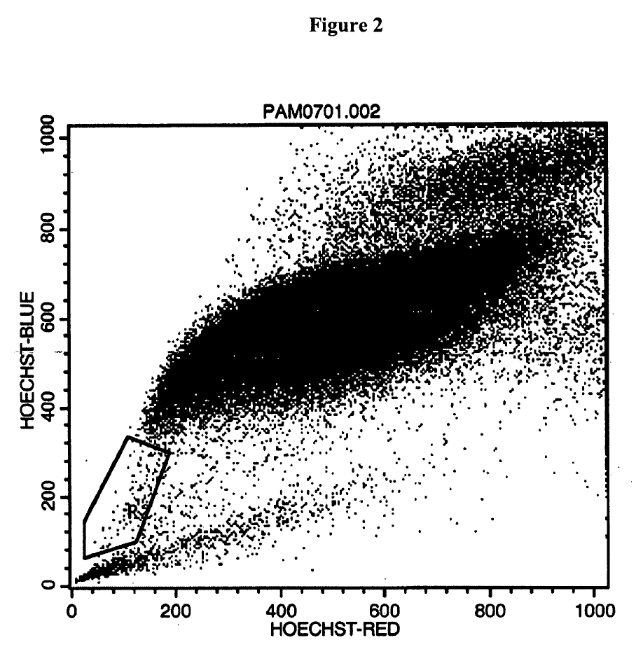

The Hoechst 33342 profiling method was used herein to identify and generate enriched dental cell side populations from cultured rat tooth bud cells. The presence of a dental cell side population in the Hoechst dye profile was used as another marker for identifying dental precursor cells. The Hoechst 33342 profiling of the cultured rat tooth bud cells (described above) is shown in FIG. 2. The cells were sorted based on their forward and side light scatter characteristics. The dental cell side populations found in the rat tooth bud cells are shown in the boxed area in FIG. 2. This SP sort resulted in the isolation of 1,300 SP cells out of a total of 4 million rat tooth bud primary cell cultures (0.0325% SP cells). Another SP sort of approximately 5 million first passage rat tooth bud cells resulted in the isolation of 10,000 SP cells (0.2%) and 75,000 non-SP cells (1.5%). These results suggest that the percentage of dental precursor cells increased in the passaged cell population. Another SP sort of 11.6 million primary rat tooth bud cell cultures, identified 10,000 SP cells (0.086%) and approximately 1 million non-SP cells (8.62%). These identified SPs contained dental epithelial and dental mesenchymal SP cells that exhibit distinct epithelial and mesenchymal cell phenotypes.

The stem cell phenotype was confirmed by staining with an antibody specific for the stem cell marker STRO-1⁺. The tooth bud-derived dental stem cells are negative for Hoechst 33324 dye and positive for STRO-1⁺ staining. Optionally, the epithelial and mesenchymal cell layers of the tooth bud are dissected and segregated prior to identification and sorting of the cells by virtue of their staining profiles.

Establishment of Clonal Dental Epithelial and Mesenchymal Side Populations

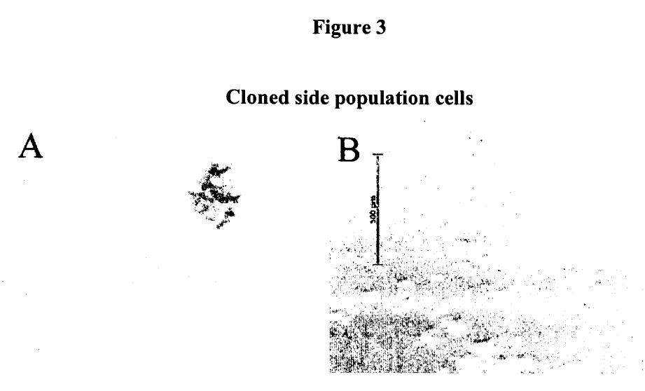

The dental epithelial and mesenchymal side population cells were cloned and expanded. Cultured enamel organ and pulp organ cells were sorted and cloned. After two weeks, distinct mesenchymal clones (FIG. 3A) and epithelial clones (FIG. 3B) were apparent. These results demonstrate that single cell suspensions of cultured epithelial and mesenchymal dental cells can be used for tooth tissue engineering.

Clonal epithelial and mesenchymal dental SP cells are further expanded and subject to molecular and/or cellular characterizations and empirical testing in a tooth tissue engineering assay.

### Example 2

### Biocompatible Implants

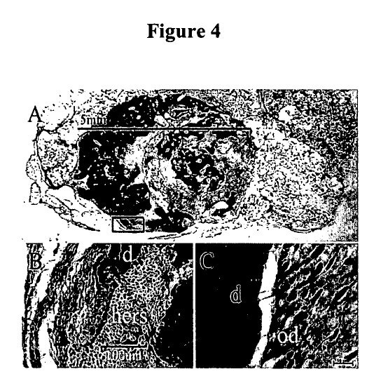

Cultured pig tooth bud cells were seeded onto a scaffold at a cell density of 7.0×10⁵ cells/mm³. The cell/scaffold construct was then implanted at a highly vascularized site, the omentum, of a mammalian host. Omentum surgeries were performed as previously described. (Young et al., J. Dent. Res., 81:695-700 (2002)). Implantation resulted in the formation of mixed bioengineered dentin and enamel tissues that approximated the size of the scaffold (FIG. 4). After a period of time, e.g., 18-20 weeks post-implantation, mature tooth tissue and de novo tooth development was observed. De novo tooth development after implantation of the cell/scaffold construct was used as another marker of dental precursor cells. Detection of de novo tooth development throughout the life of the omental implant suggests the in vivo presence and maintenance of these dental precursor cells.

### Example 3

### Detection of Dentin/Enamel Junction (DEJ) Formation

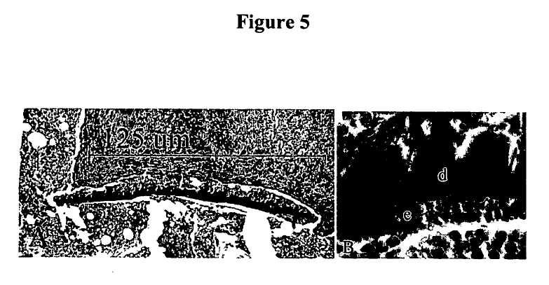

Enamel organ and pulp organ tissues were isolated and used to generate single cell suspensions of dental epithelial or mesenchymal cells, respectively, as described above in Example 1. A first scaffold was seeded either with 1.5×10⁷ enamel epithelial cells, and a second scaffold was seeded with 1.0×10⁷ pulp mesenchymal cells. These scaffolds were sutured together, implanted in the omentum of nude rat hosts, and grown for 8 weeks. Histological analyses of harvested implant tissue revealed adjacent layers of dentin and enamel that aligned with the interface of the two scaffolds (FIG. 5, Panel A, scaffold interface is indicated by red dotted line). No scaffold-guided DEJs were observed in scaffolds seeded with mixed epithelial and mesenchymal dental cell populations. In addition, scaffold implants seeded with dental mesenchymal cells alone formed osteodentin, and implants seeded with dental epithelial cells alone did not form enamel. These results indicate that, as is the case with natural tooth development, epithelial and mesenchymal dental cell interactions are required to form bioengineered tooth structures. Thus, bioengineered tooth development is characteristic of natural tooth development.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising an isolated dental epithelial precursor cell.

2. The composition of claim 1, wherein said dental epithelial precursor cell is STRO-1⁺ and comprise a low level of Hoechst 33324 dye in a Hoechst 33324 profile.

3. The composition of claim 1, wherein said dental epithelial precursor cell differentiates into an enamel-producing cell.

4. A composition comprising an isolated dental mesenchymal precursor cell.

5. The composition of claim 4, wherein said dental mesenchymal precursor cell is STRO-1⁺ and comprise a low level of Hoechst 33324 dye in a Hoechst 33324 profile.

6. The composition of claim 4, wherein said dental mesenchymal precursor cell differentiates into a dentin-producing cell.

7. An isolated population of cells comprising a mixture of isolated dental epithelial precursor cells and isolated dental mesenchymal precursor cells.

8. The population of claim 7, wherein said dental epithelial precursor cells differentiate into enamel-producing cells and said dental mesenchymal precursor cells differentiate into dentin-producing cells, further wherein each of said dental epithelial precursor cells and said dental mesenchymal precursor cells is STRO-1⁺ and each of said dental epithelial precursor cells and said dental mesenchymal precursor cells comprise a low level of a detectable label in a Hoechst dye profile.

9. The composition of claim 1, wherein said cell is obtained from a post-natal tooth bud.

10. The composition of claim 4, wherein said cell is obtained from a tooth pulp tissue or a post-natal tooth bud.

11. The composition of claim 7, wherein said cells are obtained from a tooth pulp tissue or a post-natal tooth bud.

12. A biocompatible implant comprising a biocompatible scaffold and the composition of claim 1.

13. A biocompatible implant comprising a biocompatible scaffold and the composition of claim 4.

14. A biocompatible implant comprising a biocompatible scaffold and the composition of claim 7.

15. A composition comprising an acellular scaffold, a first population of cells, a second population of cells, and an interface between said first population and said second population, wherein said first population comprises isolated dental mesenchymal precursor cells and said second population comprises isolated dental epithelial precursor cells and wherein said first population and said second population are spatially segregated on said scaffold.

16. The composition of claim 15, wherein said interface comprises a contact between said first population and said second population.

17. The composition of claim 15, further comprising enamel at said interface between said first population and said second population.

18. A biocompatible implant comprising:
(a) a first biocompatible scaffold and the composition of claim 1; and
(b) a second biocompatible scaffold and the composition of claim 4,
wherein at least a portion of said first biocompatible implant is in contact with at least a portion of said second biocompatible implant, thereby forming an interface between said first and second biocompatible scaffolds.

19. The biocompatible implant of claim 18, wherein said first biocompatible scaffold is located within the biocompatible implant at a position that is inferior to the location of said second biocompatible scaffold, such that at least a portion of said interface between said first and second biocompatible scaffolds produces a dentin/enamel junction.

20. A method for generating tooth tissue in a subject comprising implanting the biocompatible implant of claim 12 at a vascularized site in a subject.

21. The method of claim 20, wherein said vascularized site of implantation is at least partially located in omental tissue of said subject.

22. A method for generating tooth tissue in a subject comprising implanting the biocompatible implant of claim 13 at a vascularized site in a subject.

23. The method of claim 22, wherein said vascularized site of implantation is at least partially located in omental tissue of said subject.

24. A method for generating tooth tissue in a subject comprising implanting the biocompatible implant of claim 14 at a vascularized site in a subject.

25. The method of claim 24, wherein said vascularized site of implantation is at least partially located in omental tissue of said subject.
